(19) United States
(12) Patent Application Publication
(10) Pub. No.: US 2019/0022271 A1
(43) Pub. Date: Jan. 24, 2019

(54) SUTURE MATERIAL HAVING BENEFICIAL PHYSICAL PROPERTIES

(71) Applicant: Riverpoint Medical, LLC, Portland, OR (US)

(72) Inventors: Patrick Edward Ferguson, Portland, OR (US); Patrick Joseph Ferguson, Portland, OR (US)

(73) Assignee: Riverpoint Medical, LLC, Portland, OR (US)

(21) Appl. No.: 16/141,364

(22) Filed: Sep. 25, 2018

Related U.S. Application Data
(63) Continuation of application No. 14/207,866, filed on Mar. 13, 2014, now abandoned.

(51) Int. Cl. A61L 17/10; A61L 17/12

(52) U.S. Cl. CPC A61L 17/105; A61L 17/12; A61L 17/10

(57) ABSTRACT

A suture that includes an exterior braid of more than 50% ultra-high molecular weight polyethylene fibers, defining a lumen having a lumen diameter. In addition, the suture material includes an interior core, comprised of fibers and having a diameter that is less than 55% of the diameter of said lumen, and wherein the interior core includes four or fewer fibers.

### RELATED APPLICATIONS

This application is a continuation of U.S. application Ser. No. 14/207,866 filed on Mar. 3, 2014 which is incorporated by reference as if fully set forth herein.

### BACKGROUND

Suture material is preferably strong and can be easily formed into durable knots. Some stiffness is also desirable, as sutures must sometimes be threaded through holes, and it is easier to push a material that has some stiffness.

Suture material typically includes an outer braid and an inner braided or twisted core of fibers. Ultra-high molecular weight polyethylene (UHMWPE), due to its exceptional strength, is a popular material for both outer braid and inner core. Although this construction appears to be the most popular for suture material, at least two problems have been noted.

First, UHMWPE is very strong, but also very hard. Accordingly, when tied in a knot, very hard material of a first fiber has a tendency to cut through a second fiber into which the first fiber is pressed, in the knot. It appears that the hard inner core may play a role by providing a hard backing to the outer braid, permitting a contacting fiber to cut through more easily, just as a cutting board permits a knife to more easily cut through a fibrous vegetable.

Second, it has been noted that the inner core is bulky and causes knots formed in the suture to have a higher profile and take up a greater volume. Anything taking up space inside the body can potentially conflict with tissue growing back and attempting to claim some of the same volume. The core of a suture, in particular, may interfere with tissue ingrowth, which is a health promoting process.

Although a suture with an outer braid alone addresses some of these problems, it does so at a price of reduced stiffness. It has a tendency to fold on itself in a somewhat unpredictable manner, making surgical handling more difficult.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a suture that includes an exterior braid of more than 50% ultra-high molecular weight polyethylene fibers, defining a lumen having a lumen diameter. In addition, the suture material includes an interior core, comprised of fibers and having a diameter that is less than 55% of the diameter of said lumen, and wherein the interior core includes four or fewer fibers.

In a second separate aspect, the present invention may take the form of a suture that includes an exterior braid of more than 50% ultra-high molecular weight polyethylene fibers, defining a lumen. Also, the suture material includes an interior core comprised of fibers, at least some of which are bio-absorbable fibers.

In addition to the exemplary aspects and embodiments described above, further aspects and embodiments will become apparent by reference to the drawings and by study of the following detailed descriptions.

### BRIEF DESCRIPTION OF THE DRAWINGS

FIG. 1 is a schematic cross-section view of suture material according to the present invention.

FIG. 2 is a side view drawing of the braided exterior of a suture.

Exemplary embodiments are illustrated in referenced drawings. It is intended that the embodiments and figures disclosed herein are to be considered illustrative rather than restrictive.

### DETAILED DESCRIPTION OF THE PREFERRED EMBODIMENT

### Definitions

“Filament” is a single strand of material.

“Fiber” is an industry term of art, meaning an element of a braid or of a group that is twisted together. One fiber may include a collection of filaments.

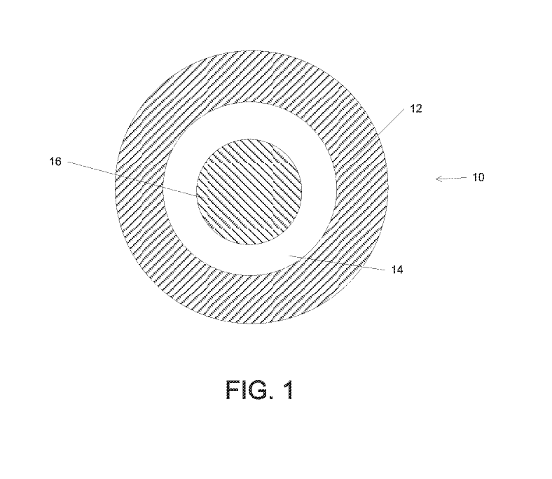

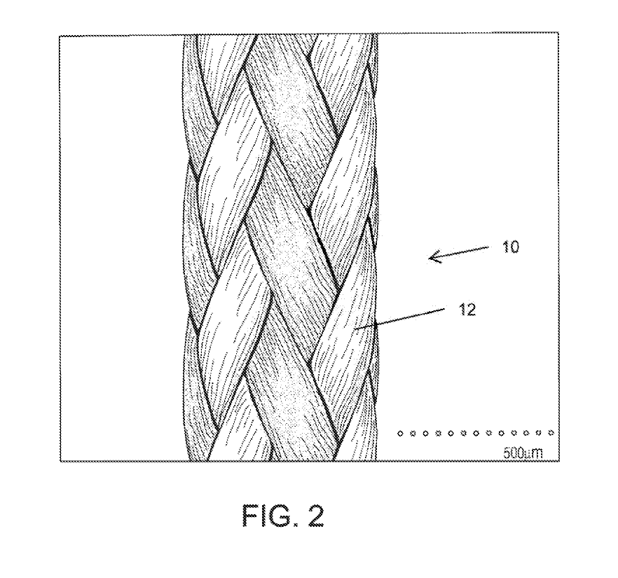

Referring to FIG. 1, in a preferred embodiment a suture 10 comprises an outer braid 12 defining a lumen 14 containing an inner core 16. The diameter of the inner core 16 is substantially less than that of the lumen 14, thereby permitting lower profile and volume knots to be formed in the suture material, compared with suture material having a core that fills the lumen. But core 16 helps to prevent the otherwise hollow outer braid from unpredictably folding over on itself and generally increases the stiffness of the suture 10, thereby improving handling qualities. FIG. 2 shows a side view of outer braid 12.

In various preferred embodiments the diameter of inner core 16 is 55%, 50%, 45%, 40% or less than the diameter of the lumen 14 (which is equal to the inner diameter of braid 12). The core may be made of a single fiber, two fibers twisted together, three or four or more fibers. Also, the core 16 may be made of UHMWPE fibers twisted, air tangled or braided with fibers of poly ester or polyethylene terephthalate both of which are more elastic than UHMWPE and will therefore not break, but stretch slightly and continue to help the overall suture 10 bear a load under stress. The smaller the core 16 is in comparison with the lumen 14 width and the greater percentage of core 16 material that is softer and more elastic and flexible than UHMWPE, the less core 16 interferes with knot tying and the lower the knot profile that is achievable. But a smaller diameter core 16 that has a higher percentage of softer material will also contribute less to the strength and stiffness of the suture. Accordingly, in one embodiment, a system of grading is established, where suture material is rated according to stiffness, strength and knotting characteristics.

In another alternative embodiment the core 16 may be made at least in part of fibers of a bio absorbable material, such as poly butyl succinate (PBS); polydioxanone (PDO); poly glycolic acid (PGA); poly lactic acid (PLA); poly glycolic-co-lactic acid (PGLA); or poly glycolide-co-caprolactone (PGCL). These materials also can accept a dye and, accordingly, can be used as tracer elements on the suture exterior braid 12, to help distinguish one suture from another. The bio-absorbable material permits the suture to shrink in dimension as it remains in the body, as the fibers of bio absorbable material are gradually absorbed. As new body tissue grows after a surgery in which sutures are placed, the full strength of the original suture is no longer needed as a portion of the stress on the suture is taken up by the newly formed body tissue. This process is beneficial to the patient, as foreign material left in the body is generally harmful to a patient.

In one preferred embodiment of the suture material 10, the fibers of the core 16 are air tangled together, in a known technique of blowing air into the fibers as they are brought into proximity. In alternative preferred embodiments they are twisted together or braided together.

The sutures described above, in which a core 16 fills only a portion of a lumen defined by a braided exterior, may be made in size 0 to size 10, or larger, the limitation being that for the smallest size sutures, it may be difficult to define a lumen and there may be little space for any space about a core. The suture material described herein may be produced on a maypole braiding machine having 4, 8, 12, 16 or 32 carriers. The air entangled core suture embodiment and the partially bio-absorbable core suture embodiment may be made in any size larger than 3-0.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A suture, comprising:
(a) an exterior braid of more than 50% ultra-high molecular weight polyethylene fibers, defining a lumen having a lumen diameter;
(b) an interior core, comprised of fibers and having a diameter that is less than 55% of the diameter of said lumen; and
(c) wherein said core includes four or fewer fibers.

2. The suture of claim 1, wherein said core is composed, at least in part, of fibers of polyester.

3. The suture of claim 1, wherein less than 60% of the fibers of said core are composed of ultra-high molecular weight polyethylene.

4. The suture of claim 1, wherein said diameter of said core is less than 50% of said lumen diameter.

5. The suture of claim 1, wherein said core is composed at least in part of bio-absorbable fibers.

6. The suture of claim 6, wherein said bio-absorbable fibers are of polybutyl succinate.

7. The suture of claim 1, wherein said core includes no ultra-high molecular weight polyethylene.

8. The suture of claim 7, wherein said core consists of polyester.

9. The suture of claim 1, wherein said core makes up less than 20% of said suture structure by weight.

10. The suture of claim 1, wherein said exterior braid is comprised of 32 fibers.

11. The suture of claim 1, wherein said exterior braid is comprised of 16 fibers.

12. The suture of claim 1, wherein said exterior braid is comprised of 8 fibers.

13. The suture of claim 1, wherein said core fibers are twisted together.

14. The suture of claim 1, wherein said core includes a single fiber, only.

15. A suture, comprising
(a) an exterior braid of more than 50% ultra-high molecular weight polyethylene fibers, defining a lumen; and
(b) an interior core comprised of fibers, at least some of which are made of poly butyl succinate.

16. The suture of claim 15, further including a bioabsorbable suture in said exterior braid, said bioabsorbable suture being dyed a distinctive color, to help identify said suture.

17. The suture of claim 15, wherein said fibers of said interior core are partially absorbed into a body.
